(19) United States
(12) Patent Application Publication
(10) Pub. No.: US 2019/0198164 A1
(43) Pub. Date: Jun. 27, 2019

(54) PATIENT-CENTRIC TIMELINE FOR MEDICAL STUDIES

(71) Applicant: INTERNATIONAL BUSINESS MACHINES CORPORATION, Armonk, NY (US)

(72) Inventors: Omid Bonakdar Sakhi, North York (CA); Rebecca Trunnell-Hyman, Armonk, NY (US); Hans Harald Zachmann, Toronto (CA); Moshe M. Gadish, Armonk, NY (US); Muhammad Talha Malik, Toronto (CA)

(21) Appl. No.: 15/855,797

(22) Filed: Dec. 27, 2017

(51) Int. Cl. G16H 40/63; G16H 30/20; G16H 70/00; G06K 9/66; G06K 9/62; G06F 3/0484

(52) U.S. Cl. CPC G16H 40/63; G16H 30/20; G16H 70/00; G06F 17/30964; G06K 9/6202; G06F 3/04842; G06F 3/04847; G06K 9/66

(57) ABSTRACT

Systems and methods for displaying medical studies. A system includes an electronic processor configured to receive a reference medical study, of a patient, having a reference modality identifier and a reference procedure type in metadata of the reference medical study; access additional medical studies of the patient, each additional medical study having a modality identifier, a procedure type, and a study time in metadata of additional medical studies; apply a set of rules to additional medical studies to determine a relevancy level for each additional medical studies from a set of relevancy levels; generate and display a GUI having a patient-centric timeline including a node for each additional medical study, the node positioned along a first dimension of the timeline based on the study time of the additional medical study and positioned along a second dimension of the timeline based on the relevancy level of the additional medical study.

### FIELD

Embodiments described herein relate to methods and systems for displaying an image, and more particularly, to displaying medical studies in a patient-centric timeline.

### SUMMARY

Physicians (such as radiologists and cardiologists) and other medical professionals typically use a commercial picture archive and communication system (PACS) when reviewing medical images (for example, medical image studies or exams). PACS is a central repository for various medical image studies of different modalities. PACS provides defined workflows for reviewing and analyzing the medical images and also functions a functions as a gatekeeper for other systems that access the medical images. A modality is anything that creates an image, x-ray, echocardiogram, magnetic resonance imaging (MM), and the like. Typically, medical images come in various modalities and are stored in the PACS central repository. Additionally, patients may typically have multiple studies at different times that may or may not be related. To understand the history of a patient, physicians often need to search through prior image studies and associated reports for a patient. Depending on the number of studies associated with a patient, a physician may have to scroll through multiple different studies to manually identify a relevant study, submit specific queries to attempt to locate relevant prior studies, or a combination thereof.

To solve these and other problems, embodiments described herein provide a two-dimensional view of prior medical imaging studies, wherein the dimensions are time and relevancy. In particular, the systems and methods described herein provide a patient-centric timeline that displays prior studies of a patient (icons or nodes representing such studies), wherein the position of prior study within the timeline indicates how the prior study is compared to a reference study in chronological order. Thus, embodiments described herein provide a user interface that provides users with easy access of medical images and other medical data for a patient. The user interface may also provide various viewing options for different types of images or data associated with a patient to improve clinical efficiency and accuracy in diagnosing and treating patients.

For example, one embodiment provides a system for generating a patient centric timeline of medical studies. The system comprising an electronic processor configured to receive a selection of a reference medical study associated with a patient, the reference medical study having a reference modality identifier and a reference procedure type in metadata associated with the reference medical study, The electronic processor is also configured to access a plurality of additional medical studies associated with the patient, each of the plurality of additional medical studies having a modality identifier, a procedure type, and a study time in metadata associated with each of the plurality of additional medical studies. The electronic processor is also configured to apply a set of rules to the plurality of additional medical studies to determine a relevancy level for each of the plurality of additional medical studies from a plurality of relevancy levels. The electronic processor applies the set of rules to the plurality of additional medical studies by comparing the reference modality identifier of the reference medical study and modality identifier of each of the plurality of additional medical studies and comparing the reference procedure type of the reference medical study and the procedure type of each of the plurality of additional medical studies. The electronic processor is also configured to generate and display the graphical user interface including the patient-centric timeline, wherein the patient-centric timeline includes a node for each of the plurality of additional medical studies, the node for each of the plurality of additional medical studies positioned along a first dimension of the timeline based on the study time of the additional medical study and positioned along a second dimension of the timeline based on the relevancy level of the additional medical study.

Another embodiment provides a method for generating a patient centric timeline of medical studies. The method includes receiving, with an electronic processor, a selection of a reference medical study associated with a patient, the reference medical study having a reference modality identifier and a reference procedure type in metadata associated with the reference medical study. The method also includes accessing, with the electronic processor, a plurality of additional medical studies associated with the patient, each of the plurality of additional medical studies having a modality identifier, a procedure type, and a study time in metadata associated with each of the plurality of additional medical studies. The method also includes applying, with the electronic processor, a set of rules to the plurality of additional medical studies to determine a relevancy level for each of the plurality of additional medical studies from a plurality of relevancy levels. Applying the set of rules to the plurality of additional medical studies includes comparing the reference modality identifier of the reference medical study and modality identifier of each of the plurality of additional medical studies and comparing the reference procedure type of the reference medical study and the procedure type of each of the plurality of additional medical studies. The method also includes generating, with the electronic processor, and displaying the graphical user interface including the patient-centric timeline, wherein the patient-centric timeline includes a node for each of the plurality of additional medical studies, the node for each of the plurality of additional medical studies positioned along a first dimension of the timeline based on the study time of the additional medical study and positioned along a second dimension of the timeline based on the relevancy level of the additional medical study.

Another embodiment is directed to a non-transitory, computer-readable medium storing instructions that, when executed by an electronic processor, perform a set of functions. The set of functions includes receiving, with the electronic processor, a selection of a reference medical study associated with a patient, the reference medical study having a reference modality identifier and a reference procedure type in metadata associated with the reference medical study. The set of functions also includes accessing, with the electronic processor, a plurality of additional medical studies associated with the patient, each of the plurality of additional medical studies having a modality identifier, a procedure type, and a study time in metadata associated with each of the plurality of additional medical studies. The set of functions also includes applying, with the electronic processor, a set of rules to the plurality of additional medical studies to determine a relevancy level for each of the plurality of additional medical studies from a plurality of relevancy levels. Applying the set of rules to the plurality of additional medical studies includes comparing the reference modality identifier of the reference medical study and modality identifier of each of the plurality of additional medical studies and comparing the reference procedure type of the reference medical study and the procedure type of each of the plurality of additional medical studies. The set of functions also includes generating, with the electronic processor, and displaying the graphical user interface including the patient-centric timeline, wherein the patient-centric timeline includes a node for each of the plurality of additional medical studies, the node for each of the plurality of additional medical studies positioned along a first dimension of the timeline based on the study time of the additional medical study and positioned along a second dimension of the timeline based on the relevancy level of the additional medical study.

Other aspects of the invention will become apparent by consideration of the detailed description and accompanying drawings.

### BRIEF DESCRIPTION OF THE DRAWINGS

FIG. 1 schematically illustrates a system for providing accessing medical studies in accordance with some embodiments.

FIG. 2 is a flowchart illustrating a method for generating a graphical user interface including a patient-centric timeline of medical studies performed by the system of FIG. 1 in accordance with some embodiments.

FIGS. 3-8 illustrate graphical user interfaces including a patient-centric timeline in accordance with some embodiments.

FIGS. 9-11 is a table illustrating an example set of rules applied by the system of FIG. 1 to set relevancy levels for medical studies represented within a patient-centric timeline in accordance with some embodiments.

### DETAILED DESCRIPTION

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality. As used in the present application, “non-transitory computer-readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

As noted above, when reviewing a medical study, such as an image study, for a patient, a physician may often attempt to locate additional studies (for example, prior image studies), such as to compare a recent study with a previous study, compare studies of the same anatomy generated using different imaging modalities, or the like. Locating such studies is a tedious process and is subject to human error. Also, the more a physician has to divert his or her attention between reviewing images and other user interfaces (such as user interfaces for querying a PACS), the more eye fatigue the physician may suffer, which can impact the physician's review of medical images.

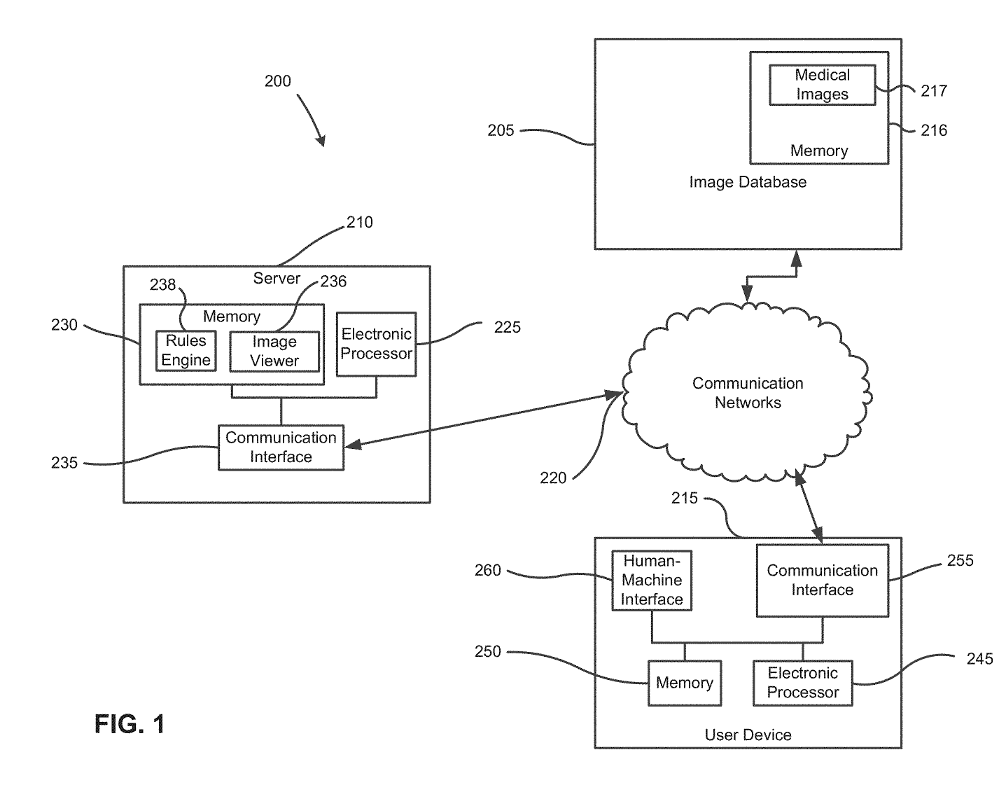

Accordingly, FIG. 1 schematically illustrates a system 200 for providing access to medical studies, such as image studies, in accordance with some embodiments. The system 200 includes an image database 205, a server 210, and a user device 215. As illustrated in FIG. 1, the image database 205, the server 210, and the user device 215 are communicatively coupled through a communication network 220. However, in other embodiments, the image database 205, the server 210, and the user device 215 communicate via one or more dedicated wire connection or other forms of wired and wireless electronic communication.

The image database 205 includes a memory 216 (a non-transitory, computer-readable medium) storing a plurality of medical images 217. In some embodiments, the image database 205 may be combined with the server 210, the user device 215, or a combination thereof. Also, in some embodiments, the medical images 217 may be stored within a plurality of databases, some of which may be included in the server 210. Although not illustrated in FIG. 1, the image database 205 may include a communication interface (similar to the communication interface included in the server 210 as described below) configured to communicate over the communication network 220. In some embodiments, the image database 205 (or a separate database included in the system 200) stores other medical data in addition to image data. For example, the image database 205 may store reports, which may be related or associated with image studies (structured report), may be laboratory reports, or the like.

The server 210 includes a plurality of electrical and electronic components that provide power, operational control, and protection of the components within the server 210. For example, as illustrated in FIG. 1, the server 210 includes an electronic processor 225, a memory 230, and a communication interface 235. The electronic processor 225, the memory 230, and the communication interface 235 are communicatively coupled via a wireless connection, a dedicated wired connection, a communication bus, or the like. Although FIG. 1 only illustrates one server 210, functionality performed by the server 210 as described herein may be distributed among multiple servers, including servers providing a cloud service. In some embodiments, the server 210 also performs functionality in addition to the functionality described herein. Further, the server 210 may further include additional components than those illustrated in FIG. 1, such as one or more human-machine interfaces.

The electronic processor 225 included in the server 210 may be a microprocessor, an application-specific integrated circuit (ASIC), or other suitable electronic device. The memory 230 includes non-transitory computer-readable medium, such as read-only memory (ROM), random access memory (RAM) (for example, dynamic RAM (DRAM), synchronous DRAM (SDRAM), and the like), electrically erasable programmable read-only memory (EEPROM), flash memory, a hard disk, a secure digital (SD) card, other suitable memory devices, or a combination thereof. The electronic processor 225 accesses and executes computer-readable instructions (“software”) stored in the memory 230. The software may include firmware, one or more applications, program data, filters, rules, one or more program modules, and other executable instructions. For example, the software may include instructions and associated data for performing a set of functions, including the methods described herein. In particular, as illustrated in FIG. 1, the memory 230 may store a timeline application 236 and a rules engine 238. As described in more detail below, the timeline application 236 (when executed by the electronic processor 225) generates a user interface that allows a user view and select medical studies, such as image studies and images included within such image studies, for display on the user device 215, including a graphical user interface that provides a patient-centric timeline as described herein. In some embodiments, the timeline application 236 provides additional functionality, such as a user interface displaying a report template for generating an electronic report, such as a radiology report for one or more medical images.

The communication interface 235 allows the server 210 to communicate with devices external to the server 210. For example, as illustrated in FIG. 1, the server 210 may communicate with the image database 205, the user device 215, and other computing resources through the communication interface 235. The communication interface 235 may include a port for receiving a wired connection to an external device (for example, a universal serial bus (USB) cable and the like), a transceiver for establishing a wireless connection to an external device (for example, over one or more communication networks 220, such as the Internet, a local area network (LAN), a wide area network (WAN), and the like), or a combination thereof.

The user device 215 may be a terminal or workstation, desktop computer, a laptop computer, a smartphone, a tablet computer, a smart television, a smart wearable, and the like. The user device 215 may include similar components as the server 210. For example, as illustrated in FIG. 1, the user device 215 includes an electronic processor 245, memory 250, and communication interface 255, which may be communicatively coupled via a wireless connection, a dedicated wired connection, a communication bus, or the like. The user device 215 also includes one or more human-machine interfaces (HMIs) 260. The human-machine interfaces 260 may include one or more input devices, such as a touch-screen, a mouse, a keyboard, a computer screen, a microphone, and the like. Although FIG. 1 only illustrates one user device 215, the system 200 may include multiple user devices. In particular, multiple user devices 215 may communicate with the server 210 to access and use the timeline application 236. In some embodiments, the user device 215 performs functionality in addition to the functionality described herein. Further, the user device 215 may further include additional components than those illustrated in FIG. 1.

A user may use the user device 215 to access medical images (and optionally other medical data) stored in the image database 205 and may access the timeline application 236 executed by the server 210 (through a browser application or a dedicated application stored and executed on the user device 215). However, in other embodiments, the medical images 217, the timeline application 236, or both are locally stored and executed by the user device 215 (the electronic processor 245).

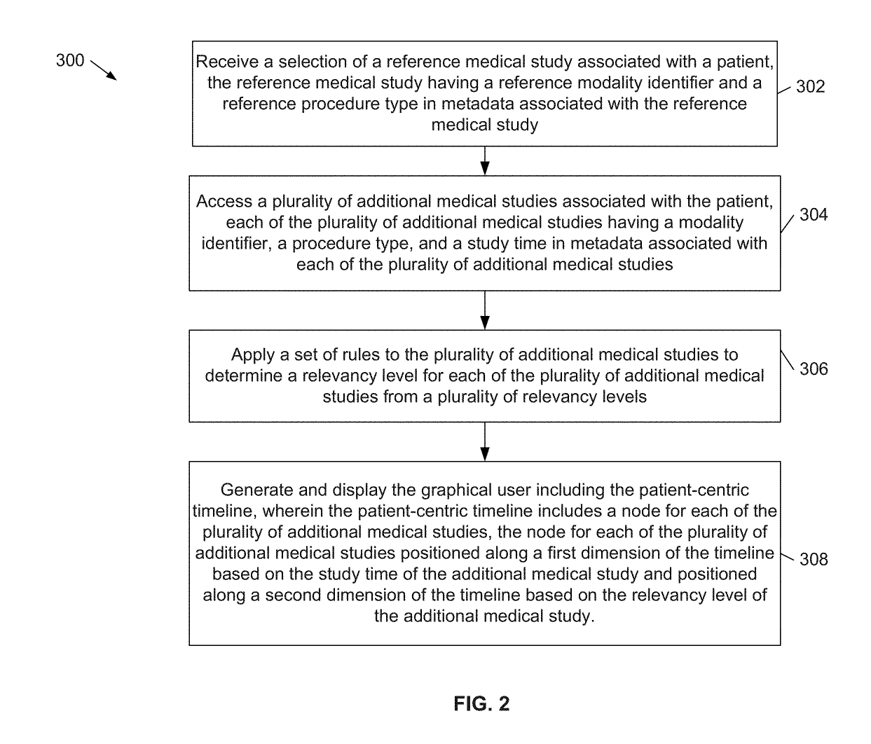

FIG. 2 is a flowchart illustrating a method 300 for generating a graphical user interface including a patient-centric timeline associated with medical studies performed by the system 200. The method 300 is described as being performed by the server 210 (by the electronic processor 225 executing the timeline application 236). However, as noted above, in some embodiments, all or a portion of the functionality included in the method 300 may be performed by the user device 215, another server, or a combination thereof.

As illustrated in FIG. 2, the method 300 includes receiving, with the electronic processor 225, a selection of a reference medical study associated with a patient (at block 302). The selection may be a manual selection of the specific reference medical study from a user (such as from a worklist of image studies awaiting review). In other embodiments, the electronic processor 225 initially receives a query from a user associated with a type of morbidity (for example, heart decease, lung infection, or the like), and the selection is based on the query results. The reference medical study includes metadata, which comprises a reference modality identifier and a reference procedure type.

As illustrated in FIG. 2, in response the selection of the reference medical study, the electronic processor 225 automatically accesses additional medical studies associated with the patient (at block 304). Each of the additional medical studies includes (as part of metadata for the studies) a modality identifier, a procedure type, and a study time. The additional medical studies may be stored in the image database 205, the server 210 (the memory 230 or a separate memory), the user device 215, within a cloud computing environment accessible via the communication network 220, on a different server, or a combination thereof.

The electronic processor 225 also automatically applies a set of rules (using the rules engine 238) to the accessed additional medical studies to determine a relevancy level for each of the additional medical studies. In some embodiments, a relevancy level is a numeric score within a predetermined range. In other embodiments, a relevancy level is a category from a predetermined set of categories, such as, for example, most relevant, relevant, least relevant and undetermined. Accordingly, the electronic processor 225 applies the set of rules (implemented via the rules engine 238) to the additional medical studies to compare (i) the reference modality identifier of the reference medical study and modality identifier of each of the plurality of additional medical studies, and (ii) the reference procedure type of the reference medical study and the procedure type of each of the plurality of additional medical studies.

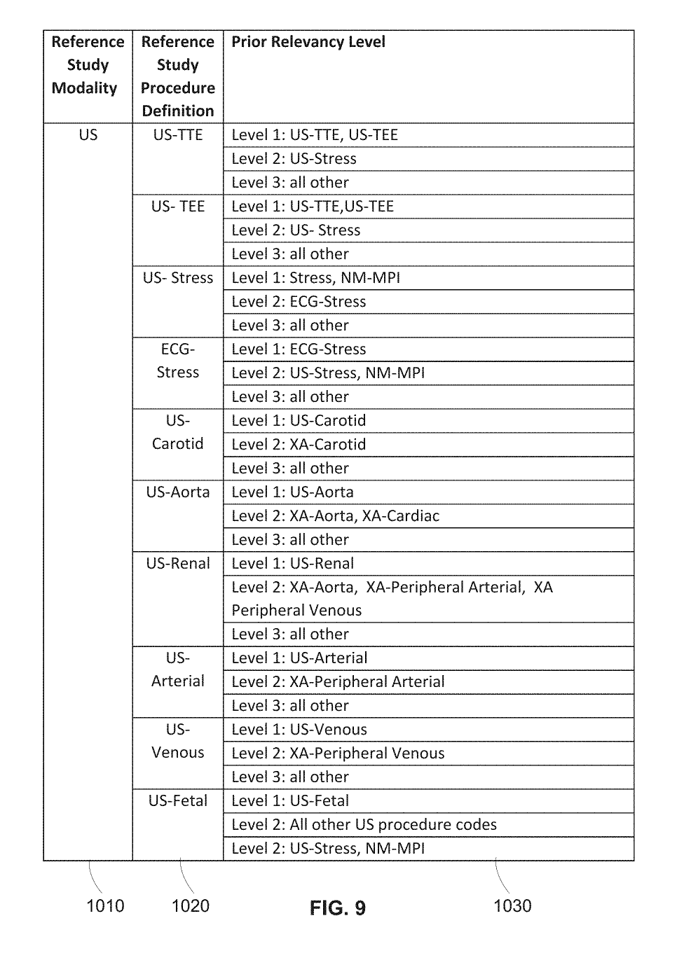

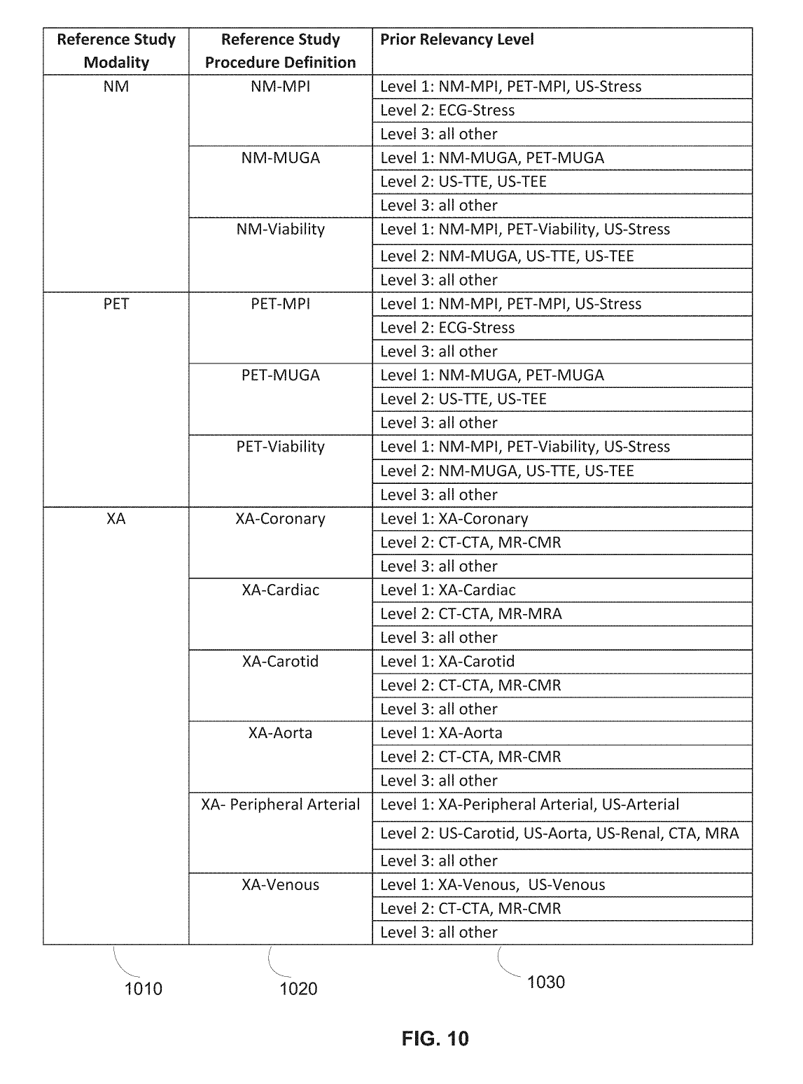

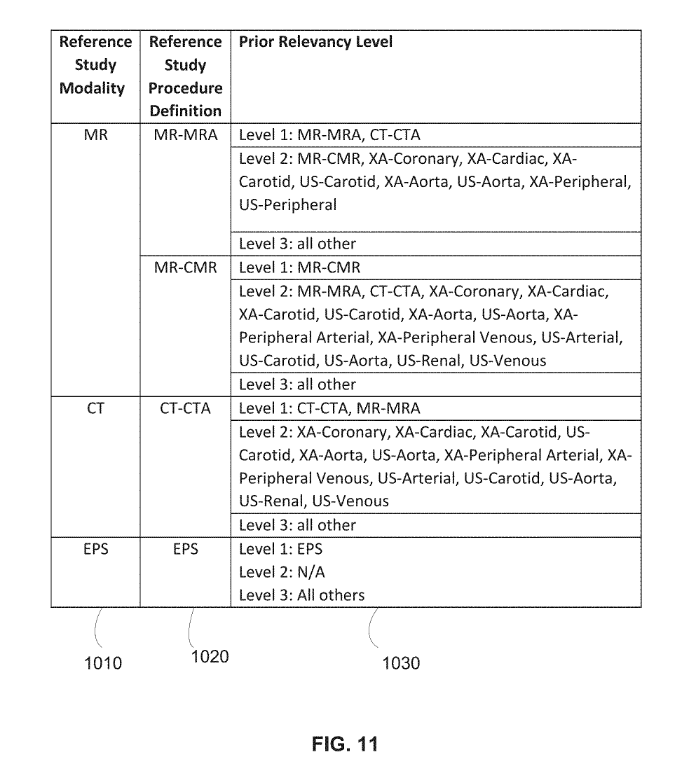

For example, FIGS. 9-11 is a table illustrating an example set of rules that may be stored in the rules engine 238. As shown in FIGS. 9-11, column 1010 identifies a particular reference study modality, column 1020 identifies a particular reference study procedure associated with a particular reference study modality (as listed in column 1010), and column 1030 identifies, for each relevancy level, zero or more modalities (and optionally procedures) for a particular reference study modality (at as listed in column 1010) and reference study procedure (as listed in column 1020). For example, as illustrated in FIG. 9, when the reference medical study is an ultrasound study (“US”) and has a procedure identifier or definition of “US-TTE,” the set of rules specify that additional medical studies that are also ultrasound studies and have a procedure identifier of “US-TTE” or “US-TEE” are assigned a relevancy level of “most relevant” or “level 1.” However, additional medical studies that are also ultrasound studies but have a procedure identifier of “US-Stress” are assigned a relevancy level of “relevant” or “level 2” and all other additional medical studies are assigned a relevancy level of “least relevant” or “level 3.” Accordingly, the set of rules can be applied to each additional medical study accessed for a reference medical study to set a relevancy level for the additional medical study.

In some embodiments, the set of rules stored in the rules engine 238 is configurable for a particular user (reviewer), group of users (a clinic, a hospital, a network, or the like), a particular patient or type of patient (patient demographics), disease or condition, imaging modality, number of additional medical studies, or the like. For example, the system 200 can provide a user interface that allows users (or an administrator) to configure the set of rules. Also, in some embodiments, the set of rules stored in the rules engine 238 is generated or customized using machine learning. Machine learning generally refers to the ability of a computer program to learn without being explicitly programmed. In some embodiments, a computer program (for example, a learning engine) is configured to construct a model (for example, one or more algorithms) based on example inputs. Supervised learning involves presenting a computer program with example inputs and their desired (for example, actual) outputs. The computer program is configured to learn a general rule (for example, a model) that maps the inputs to the outputs. The computer program may be configured to perform machine learning using various types of methods and mechanisms. For example, the computer program may perform machine learning using decision tree learning, association rule learning, artificial neural networks, inductive logic programming, support vector machines, clustering, Bayesian networks, reinforcement learning, representation learning, similarity and metric learning, sparse dictionary learning, and genetic algorithms. Using all of these approaches, a computer program may ingest, parse, and understand data and progressively refine models for data analytics. For example, a learning engine may be configured to track actions by a user to identify relevant medical studies and automatically set the rules (models) that can be applied to the reference medical study to determine a level of relevancy as described above.

With continued reference to FIG. 2, the electronic process 225 generates and displays the graphical user interface (GUI) including the patient-centric timeline (at block 308). The patient-centric timeline includes a node for each of the plurality of additional medical studies, the node for each of the plurality of additional medical studies positioned along a first dimension of the timeline based on the study time of the additional medical study and positioned along a second dimension of the timeline based on the relevancy level of the additional medical study.

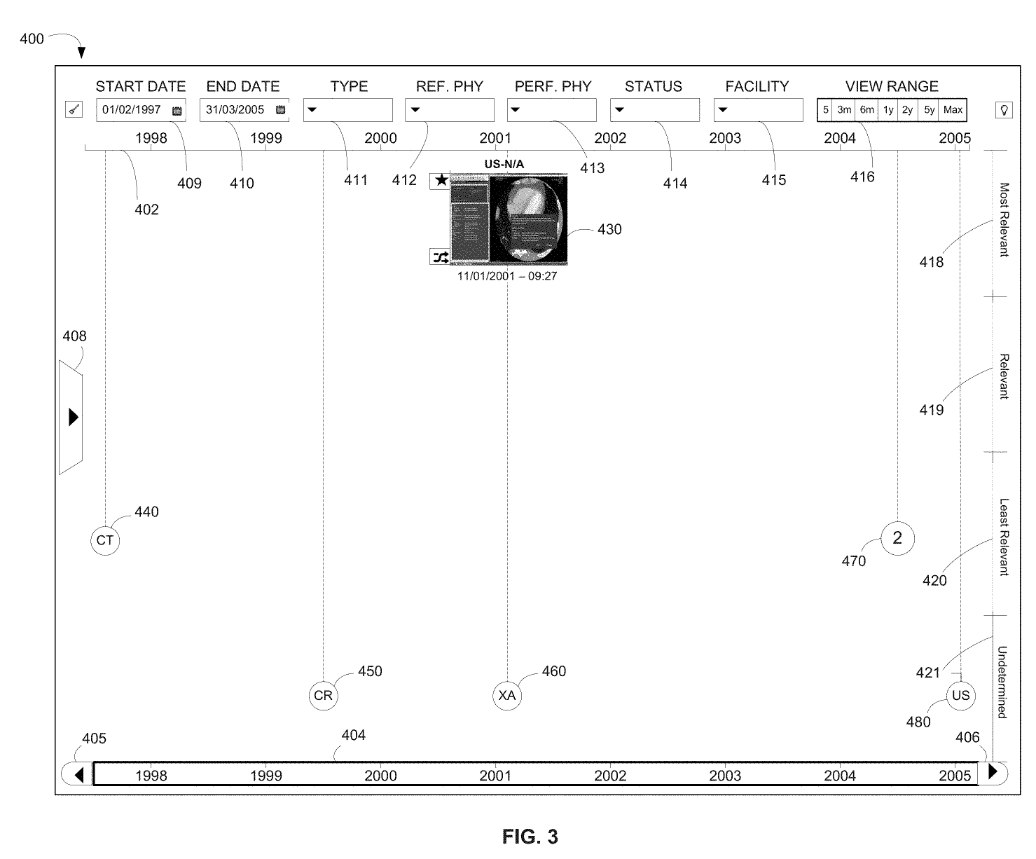

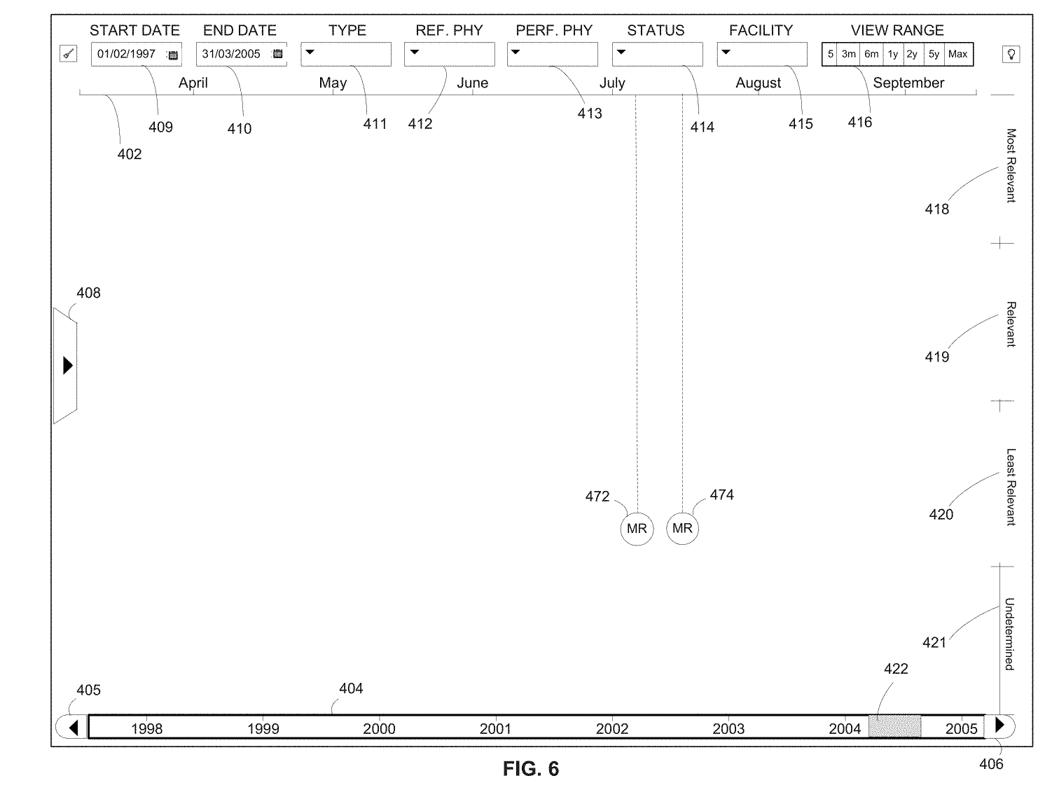

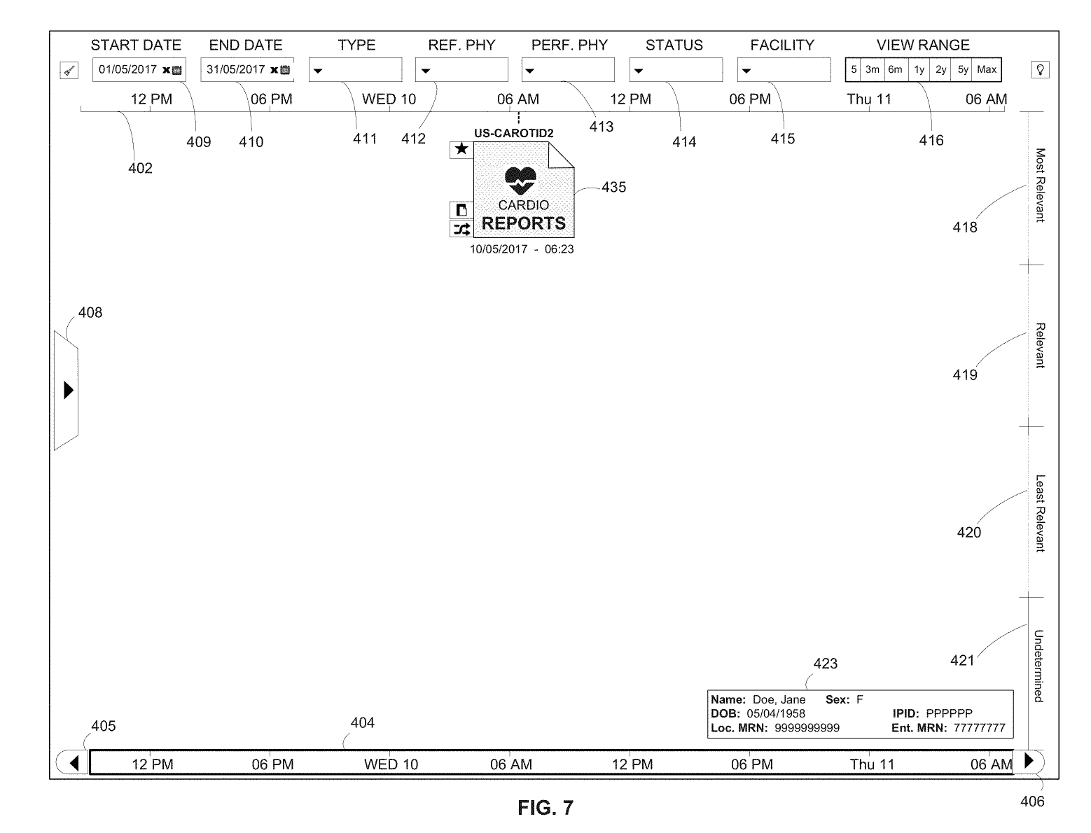

For example, FIGS. 3-8 illustrate graphical user interfaces including a patient-centric timeline in accordance with some embodiments. As shown in FIG. 3, the graphical user interface 400 includes a timeline 402 and a timeline slide bar 404 that allows several medical studies (reports, images, and the like) associated with a patient to be positioned in the graphical user interface 400. Each end of the timeline slide bar 404 includes tabs 405 and 406 that enable a user to move the timeline slide bar 404. For example, the tab 405 allows a user to shift the timeline slide bar 404 to an earlier date and the tab 406 allows the user to shift the timeline slide bar 404 to a later date. In particular, as illustrated in FIG. 6, the timeline slider 422 is movable by a user to change the window of observation for medical studies within the timeline 402. For example, in the example shown in FIG. 6, the window of observation is between March 2004 and September 2004. However, in the example shown in FIG. 7, the window of observation is between 6 AM, Wednesday, May 10, 2017 and 6 AM, Thursday, May 11, 2017. In some embodiments, a user can also zoom in and out on the timeline 402. Changing the zoom level of the timeline 402 may automatically change the start and end time (duration or window of observation) of the timeline 402.

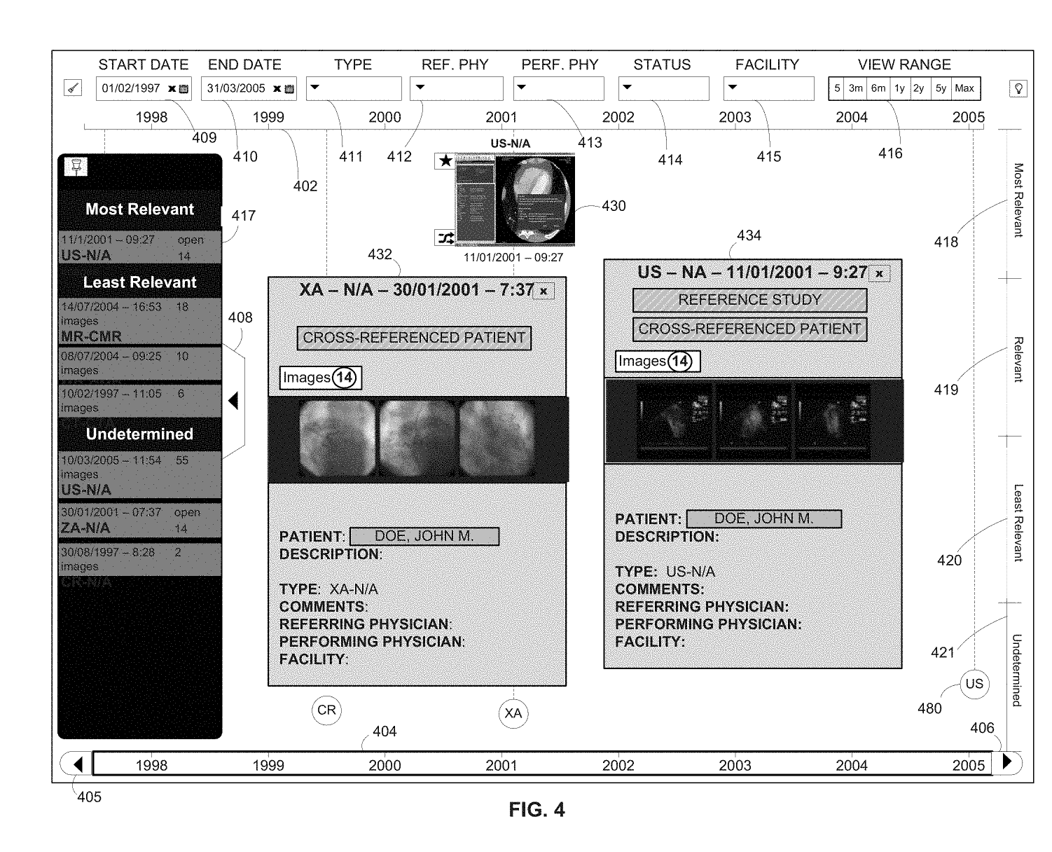

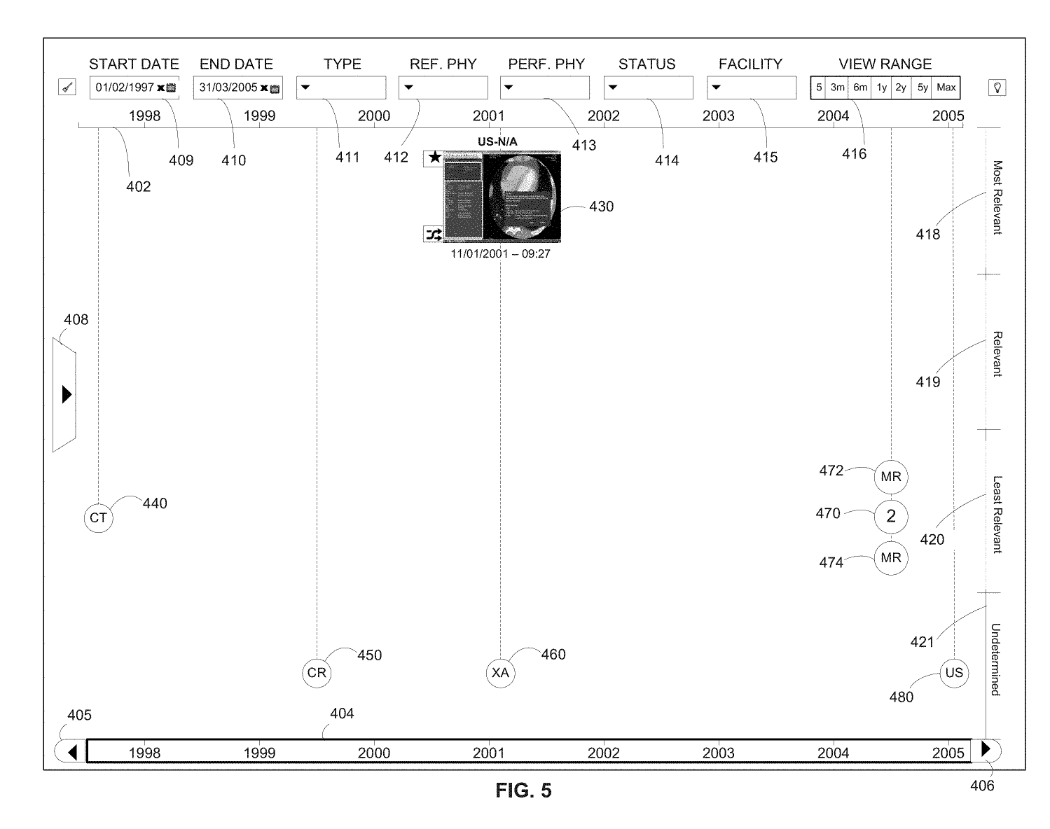

The graphical user interface 400 may also include is a sidebar selection tab 408 that allows a user to display a sidebar or hide a sidebar within the graphical user interface 400. For example, as illustrated in FIG. 4, the sidebar selection tab 408 has been selected such that the sidebar 417 is displayed in the graphical user interface 400. The sidebar 417 includes a listing of the medical studies associated with the patient that are most relevant, least relevant and undermined. A user may select a study from the listing included in the sidebar 417 to automatically zoom or adjust the patient-centric timeline to display (center) the node associated with the selected study from the listing, view an expanded display 432 of the selected medical study, or a combination thereof. As illustrated in FIG. 5, a user may also be able to select the node 430 representing the reference medical study to view an expanded display 434 of the reference medical study. As illustrated in FIG. 5, a user can open an expanded display of one or more of nodes, which allows the user to easily compare images and, optionally, generated reports between various image studies. As noted above, the particular studies that a user reviews in an expanded display or side-by-side may be tracked by a learning engine and used to develop the set of rules used to set relevancy levels are described above. In some embodiments, selecting a node may display the expanded view of the medical study associated with the node or may open the medical study in a separate, such as the native application for the type of images or data included in the medical study.

As illustrated in FIG. 3, the graphical user interface 400 also includes a start date field 409, an end date field 410, a type field 411, a referring physician field 412, a performing physician field 413, a status field 414, a facility field 415, and a view range 416. The start date field 409 and the end date field 410 may be used by the user to set a window of observation for a particular patient's various medical studies (set a duration or window of observation of the timeline displayed within the graphical user interface 400). In some embodiments, the view range 416 includes a number of user-selectable options such as 5 days, 3 months, 6 months, 1 year, 2 years, 5 years, and a maximum time, which may effectively be an infinite time range (or any time associated with an image 217 managed by the 200). The type field 411, referring physician field 412, performing physician field 413, status field 414, and facility field 415 are selectable by a user to filter the nodes included in the timeline 402. For example, a user may select a referring physician field 412 (select a particular referring physician) to limit the nodes represented in the timeline 402 to medical studies ordered by the selected referring physician.

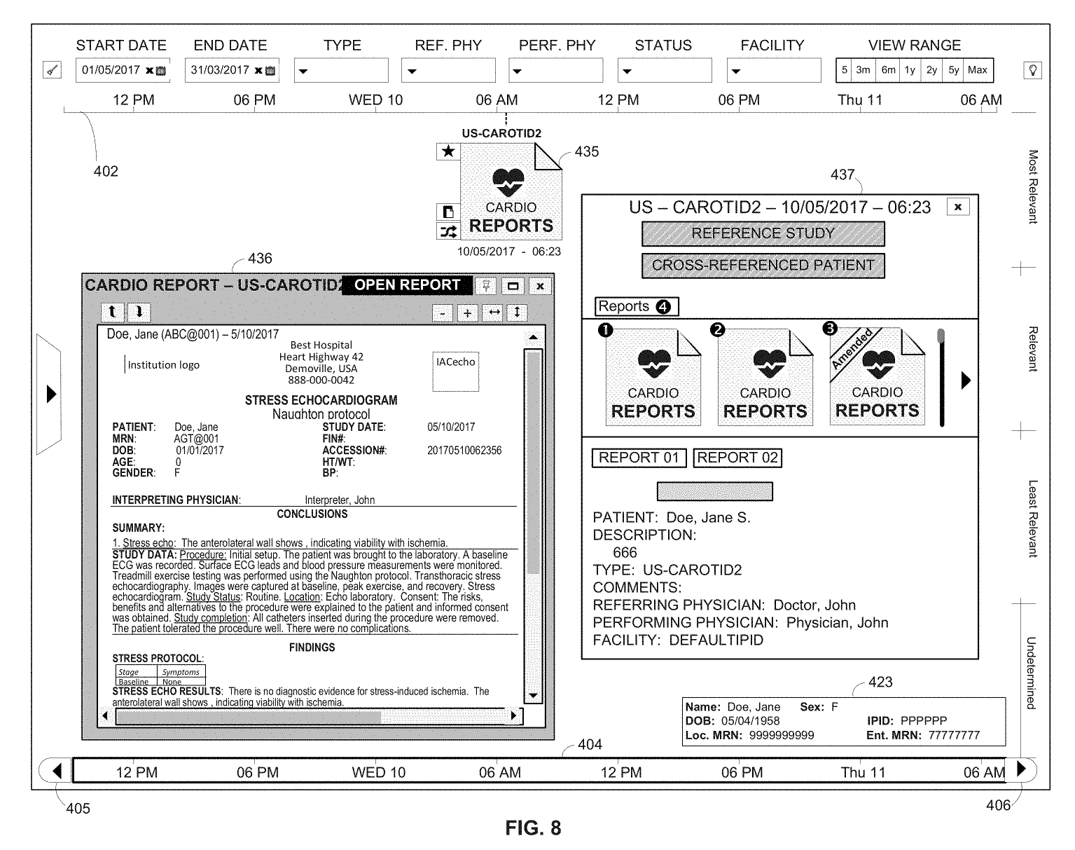

Also shown in FIG. 3, the graphical user interface 400 also displays a node 430 associated with the reference medical study of the patient and nodes 440, 450, 460 and 470 that are each associated with one of the additional medical studies accessed for the patient. The node 430 may be an icon that, when selected by a user, generates an exploded view of the medical study represented by the node 430. The node 430 also includes an indication of the date and time of the reference medical study (for example, “11/01/2001—09.27”). As illustrated in FIG. 7, the graphical user interface 400 may also include an identifier tag 423 that provides information about the patient (such as Name, Sex, Date of Birth, and various other identification details). In addition, the graphical user interface 400 and, in particular, the timeline 402, may include nodes representing reports (such as cardio repots 435) associated with the patient. Thus, the timeline 402 may include information regarding medical studies including image studies, reports associated with such image studies, reports generated separate from a medical study, including, for example, a laboratory report, or a combination thereof. For example, as illustrated in FIG. 8, the graphical user interface 400 includes a node 437 associated with a reference medical study and a node 436 associated with another medical study (for example, a stress echocardiogram) that are both associated with the patient listed in identifier tag 423.

Each of the nodes 440, 450, 460, 470, and 480 represents a particular additional medical study associated with the patient. As described above, each of the nodes 440, 450, 460, 470, and 480 are positioned in a first dimension (vertically) within the graphical user interface 400 based on the relevancy level determined for the associated additional medical study. As noted above, in some embodiments, the relevancy of a medical study is categorized as most relevant 418, relevant 419, or least relevant 420. Also, in some embodiments, when the relevancy of the medical study is unknown, the medical study may be categorized as undetermined 421. In some embodiments, the most relevant medical studies are positioned at the top of the graphical user interface 400 followed by the relevant, least relevant, and the undetermined medical studies, which are positioned at the bottom of the graphical user interface 400. For example, if the reference medical study relates to the patient's heart, an additional medical study associated with the patient that is a CT scan of the patient's brain may be identified as least relevant to the reference medical study and, hence, positioned toward the bottom of the graphical user interface 400. As shown in FIG. 5, nodes 450, 460, and 480 categorized as undetermined may be positioned at the bottom of the graphical user interface 400.

In some embodiments, each of the nodes 440, 450, 460, 470, and 480 includes an indicator (for example, “CT” indicating a “Computed Tomography”; “CR” indicating “Computed Radiography”; “XA” indicating “X-Ray Angiography”; and “US” indicating “Ultrasound;” or “MR” indicating “magnetic resonance”) that denotes a particular modality associated with the medical study. The indicator of the modality may be textual, numeric, graphical, a color, or the like.

In some embodiments, each of the nodes 440, 450, 460, 470, and 480 also includes an indicator that denotes the number of medical studies associated with a particular node. For example, node 470 includes the indicator “2” to denote that there are two medical studies associated with node 470. Thus, node 470 may be considered an aggregate node that represents multiple nodes and, hence multiple medical studies. Aggregate nodes may be used to group nodes (medical studies) depending on a duration of the timeline or a zoom level of the timeline. For example, when a patient has multiple medical studies conducted over a particular time period, the graphical user interface 400 may not have space to display a separate node for each study. Accordingly, the aggregate nodes displayed within the graphical user interface 400 may change as the duration of the timeline changes, the zoom level of the timeline changes, or the like. A user may select (click, mouse over, or the like) an aggregate node to view additional information or the individual nodes represented by the aggregate node, and a user may select an individual node to view additional information regarding the associated medical study. For example, as illustrated in FIG. 5, the graphical user interface 400 may display an expanded view of the aggregate node 470, which includes a node (472, 474) for each medical study represented by the aggregate node 470.

Thus, embodiments described herein provide methods and systems for generating a graphical user interface including a patient-centric timeline for medical studies. Various features and advantages are set forth in the following claims.

What is claimed is:

1. A system for generating a graphical user interface including a patient-centric timeline of medical studies, the system comprising:
an electronic processor configured to
receive a selection of a reference medical study associated with a patient, the reference medical study having a reference modality identifier and a reference procedure type in metadata associated with the reference medical study,
access a plurality of additional medical studies associated with the patient, each of the plurality of additional medical studies having a modality identifier, a procedure type, and a study time in metadata associated with each of the plurality of additional medical studies,
apply a set of rules to the plurality of additional medical studies to determine a relevancy level for each of the plurality of additional medical studies from a plurality of relevancy levels, wherein applying the set of rules to the plurality of additional medical studies includes
(i) comparing the reference modality identifier of the reference medical study and modality identifier of each of the plurality of additional medical studies, and
(ii) comparing the reference procedure type of the reference medical study and the procedure type of each of the plurality of additional medical studies, and
generate and display the graphical user interface including the patient-centric timeline, wherein the patient-centric timeline includes a node for each of the plurality of additional medical studies, the node for each of the plurality of additional medical studies positioned along a first dimension of the timeline based on the study time of the additional medical study and positioned along a second dimension of the timeline based on the relevancy level of the additional medical study.

2. The system of claim 1, wherein the electronic processor is further configured to automatically configure the set of rules using machine learning by tracking medical studies compared by one or more users.

3. The system of claim 1, wherein the relevancy level is selected from a group consisting of most relevant, relevant, least relevant, and undetermined.

4. The system of claim 1, wherein the electronic processor is further configured to receive a start date and an end date from a user and customize the first dimension of the patient-centric timeline based on the start date and the end date.

5. The system of claim 1, wherein the first dimension of the patient-centric timeline is based on view range selected by a user from a group consisting of five days, three months, six months, one year, two years, five years, and a maximum available range.

6. The system of claim 1, wherein the electronic processor is configured to receive a user query and receive the selection based on results of the user query.

7. The system of claim 1, wherein the electronic processor is configured to access the plurality of additional medical studies based on a manual selection of the reference medical study received from the user.

8. The system of claim 1, wherein the patient-centric timeline includes an aggregate node including a count of the one or more nodes associated with the aggregate node.

9. A method of generating a graphical user interface including a patient centric timeline for medical studies, the method comprising:
receiving, with an electronic processor, a selection of a reference medical study associated with a patient, the reference medical study having a reference modality identifier and a reference procedure type in metadata associated with the reference medical study,
accessing, with the electronic processor, a plurality of additional medical studies associated with the patient, each of the plurality of additional medical studies having a modality identifier, a procedure type, and a study time in metadata associated with each of the plurality of additional medical studies,
applying, with the electronic processor, a set of rules to the plurality of additional medical studies to determine a relevancy level for each of the plurality of additional medical studies from a plurality of relevancy levels, wherein applying the set of rules to the plurality of additional medical studies includes
(i) comparing the reference modality identifier of the reference medical study and modality identifier of each of the plurality of additional medical studies, and
(ii) comparing the reference procedure type of the reference medical study and the procedure type of each of the plurality of additional medical studies, and
generating, with the electronic processor, and displaying the graphical user interface including the patient-centric timeline, wherein the patient-centric timeline includes a node for each of the plurality of additional medical studies, the node for each of the plurality of additional medical studies positioned along a first dimension of the timeline based on the study time of the additional medical study and positioned along a second dimension of the timeline based on the relevancy level of the additional medical study.

10. The method of claim 9, further comprising automatically configuring the set of rules using machine learning by tracking medical studies compared by one or more users.

11. The method of claim 9, wherein applying the set of rules to the plurality of additional medical studies to determine a relevancy level for each of the plurality of additional medical studies includes applying the set of rules to the plurality of additional medical studies to determine the relevancy level selected from a group consisting of most relevant, relevant, least relevant, and undetermined.

12. The method of claim 9, further comprising receiving a start date and an end data and customizing a first dimension of the patient-centric timeline based on the start date and the end date.

13. The method of claim 9, further comprising customizing the first dimension of the patient-centric timeline based on a view range selected by a user from a group consisting of five days, three months, six months, one year, two years, five years, and maximum available.

14. The method of claim 9, further comprising receiving a user query and wherein receiving the selection of the reference medical study associated with the patient includes receiving the selection of the reference medical study based on results of the user query.

15. The method of claim 9, further comprising accessing the plurality of additional medical studies based on a manual selection of the reference medical study received from the user.

16. A non-transitory, computer-readable medium storing instructions that, when executed by an electronic processor, perform a set of functions, the set of functions comprising:
receiving a selection of a reference medical study associated with a patient, the reference medical study having a reference modality identifier and a reference procedure type in metadata associated with the reference medical study,
accessing a plurality of additional medical studies associated with the patient, each of the plurality of additional medical studies having a modality identifier, a procedure type, and a study time in metadata associated with each of the plurality of additional medical studies,
applying a set of rules to the plurality of additional medical studies to determine a relevancy level for each of the plurality of additional medical studies from a plurality of relevancy levels, wherein applying the set of rules to the plurality of additional medical studies includes
(i) comparing the reference modality identifier of the reference medical study and modality identifier of each of the plurality of additional medical studies, and
(ii) comparing the reference procedure type of the reference medical study and the procedure type of each of the plurality of additional medical studies, and
generating and displaying the graphical user interface including the patient-centric timeline, wherein the patient-centric timeline includes a node for each of the plurality of additional medical studies, the node for each of the plurality of additional medical studies positioned along a first dimension of the timeline based on the study time of the additional medical study and positioned along a second dimension of the timeline based on the relevancy level of the additional medical study.

17. The non-transitory, computer-readable medium of claim 16, wherein the set of functions further comprises automatically configuring the set of rules using machine learning by tracking medical studies compared by one or more users.

18. The non-transitory, computer-readable medium of claim 16, wherein the relevancy level includes at least one selected from a group consisting of most relevant, relevant, least relevant, and undetermined.

19. The non-transitory, computer-readable medium of claim 16, wherein the set of functions further comprises receiving a start date and an end date and customizing the first dimension of the patient-centric timeline based on the start date and the end date.

20. The non-transitory, computer-readable medium of claim 16, wherein the set of functions further comprises customizing the patient-centric timeline based on a view range selected by a user from a group consisting of five days, three months, six months, one year, two years, five years, and maximum available.
